(19) United States
(12) Patent Application Publication
(10) Pub. No.: US 2025/0268465 A1
(43) Pub. Date: Aug. 28, 2025

(54) VISUAL OBJECT, METHOD AND COMPUTER PROGRAM PRODUCT FOR DETERMINING ONE OR MORE VISUAL PROPERTIES OF A TEST SUBJECT

(71) Applicant: Rodenstock GmbH, Muenchen (DE)

(72) Inventors: Adam Muschielok, Miesbach (DE); Anne Seidemann, Muenchen (DE)

(21) Appl. No.: 18/862,281

(22) PCT Filed: Apr. 26, 2023
(86) PCT No.: PCT/EP2023/060955

§ 371 Date: Nov. 1, 2024

(30) Foreign Application Priority Data
May 3, 2022 (DE) 102022001541.2

(51) Int. Cl. A61B 3/032; A61B 3/02; A61B 3/09; G06T 5/20; G06T 5/50

(52) U.S. Cl. CPC A61B 3/032; A61B 3/022; A61B 3/09; G06T 5/20; G06T 5/50; G06T 2207/20212; G06T 2207/30041

(57) ABSTRACT

A visual object for determining one or more visual properties of a test person. The visual object includes at least two different filtered images combined to form a test image, wherein each of the filtered images represents one of at least two predetermined different original images and is provided with a filter specific to a predetermined optically induced blur.

### TECHNICAL FIELD

The invention relates to a visual object as well as a method and a computer program product for determining one or more visual properties of a test person. In particular, the invention can be assigned to the field of optometry.

### BACKGROUND

To determine visual properties, i.e. eyesight and/or certain visual functions (such as visual acuity, contrast perception, binocular vision, spatial and temporal resolution, oculomotor system, accommodation, adaptation, twilight vision, daytime vision, etc.), of a test person or proband, measurement methods using visual objects are used. For example, to determine vision, the test person is presented periodic patterns or standardized representations he or she is to recognize and identify on a test surface (such as a board, a screen or a projection wall).

Conventionally, eye test symbols, also referred to as optotypes, are mainly used. Such optotypes are usually graphic symbols (e.g. numbers, letters and/or figures) that are used to determine visual acuity, for example. Well-known optotypes include the Snellen letter, the Snellen hook or the Landolt ring. The decisive factor for an optotype is that it can be clearly identified or named by the test persons.

If optotypes are to be recognized with optically induced blur (e.g. due to defocus and/or astigmatism and/or higher-order aberrations)—e.g. as part of an optometric examination—this is often difficult as the blur of at least one of the test person's eyes increases, because the test person cannot assign the optotype he's seeing in a blurred fashion to an object that is easy to name.

Usually, a test person recognizes ordinary optotypes (e.g. Landolt rings, numbers or letters) less well when viewed with optically induced blur than without optically induced blur. Thus, a test person with optically induced blur may potentially need a long time to classify an optotype.

EP 2 243 419 B1 describes visual objects or optotypes the average luminance of which is substantially equal to the luminance of the background. The optotypes disclosed in EP 2 243 419 B1 are either seen as a whole or not seen depending on the fineness of the lines when viewed with an optically induced blur, and the test person must report which or how many objects were seen. It is therefore not sufficient here to name a single optotype, which can lead to errors or inaccuracies in the determination of visual properties.

It is therefore an object of the present invention to provide a visual object and a method for a simple, reliable and fast determination of the visual properties of a test person. This object is solved by the subject matters of the independent claims. Advantageous embodiments are subject of the subclaims.

A first independent aspect for solving the object relates to a visual object for determining one or more visual properties of a test person, comprising at least two different filtered images combined (in particular superimposed) to form a test image, wherein each of the filtered images represents one of at least two predetermined different original images and is provided with a filter specific to a predetermined optically induced blur. In particular, each filtered image is assigned to an original image and a filter or a filter function. In particular, each of the filtered images represents one of at least two predetermined different original images. In particular, each of the filtered images is provided with a filter associated with or assigned to its represented original image and specific to a predetermined optically induced blur.

The visual object is in particular presented using conventional means including a projection onto the retina of a test person (in particular using means suitable for determining refraction, such as refractive or diffractive lenses and a screen). In other words, the visual object can be presented by technically common methods such as by appropriately designed items (in particular a printout of the visual object, e.g. on paper), by front-lit or back-lit panels, by a projection onto a board or a wall, by displays on a screen or monitor, and by a projection directly onto the retina of at least one eye of the test person.

The term “visual properties” of a test person particularly includes the test person's eyesight and/or various visual functions of the test person. For example, with the help of one or more visual objects according to the invention, values or limit values for visual acuity, contrast perception or contrast sensitivity, binocular and stereo vision, adaptability or adaptation speed, etc. can be determined or identified. In particular, the visual object and the method described herein serve to measure and/or optimize the test person's eyesight.

The “test image” is understood to mean in particular the overall image that combines and/or superimposes the individual filtered images. The filtered images are based on at least two predetermined different original images. In particular, more than two (e.g. three, four, five, six, etc.) original images or a plurality of original images can be predetermined. In the context of this invention, the “original image” refers in particular to an original, unfiltered image. An original image can e.g. be one or more symbols, optotypes, graphics or other objects that are easily and clearly recognizable for a test person. In this context, “objects” are understood to mean not only symbols or objects, but also plants, animals, persons or figures. For example, an original image can comprise or include a photo or an image of an item familiar to the test person, a person known to the test person or a cartoon character known to the test person. The predetermined original images differ from one another, i.e. each of the predetermined original images includes or shows a different, clearly recognizable or identifiable object.

Each filtered image represents an original image, i.e. each filtered image is assigned an original image (from the predetermined original images). Each filtered image is associated with one of the predetermined original images. In other words, the original image represented by a filtered image is therefore an original image associated with the filtered image.

Each of the filtered images is provided with a filter or a filter function. This means that the original image represented by the filtered image or associated with the filtered image is filtered with a respective filter or filter function to thereby generate the filtered image. In other words, the corresponding filter or filter function is applied to the original image.

A filter or a filter function is specific to a predetermined (in particular specified and/or predefined) optically induced blur. In the context of this invention, “optically induced blur” is to be understood in particular as the blur of the image of an object (e.g. the visual object or the test image) on the retina of the at least one eye of the test person. When an object is imaged on the retina of the at least one eye of the test person, the entire imaging process is taken into account, i.e. imaging through the at least one eye of the test person and, if applicable, additionally imaging through any other optical system (e.g. refractive glasses, a contact lens, a phoropter, and/or other lenses) which, depending on the application, may be arranged in front of the at least one eye of the test person (i.e. between the at least one eye of the test person and the object). If no other optical system is arranged in front of the at least one eye of the test person, only imaging through the at least one eye of the test person is taken into account. The entire imaging process, which produces the “optically induced blur” or the blur of the image of the object on the retina of at least one eye of the test person, therefore takes into account a possible (i.e. any) vision defect of at least one eye of the test person as well as a possible (i.e. any) additional optical system (e.g. refractive glasses, contact lenses, phoropters, and/or other lenses) arranged in front of at least one eye of the test person. Furthermore, the pupil size of at least one eye of the test person can also be taken into account in the imaging process. In particular, the “optically induced blur” of the test person is based on a possibly present vision defect of at least one eye of the test person, the pupil size of at least one eye of the test person, and possibly on (any) additional optical system arranged in front of at least one eye of the test person. The “optically induced blur” can include or be an intrinsic blur of at least one eye of the test person (referred to briefly as “intrinsically induced blur” within the scope of the invention) and/or an extrinsic optically induced blur (referred to briefly as “extrinsically induced blur” within the scope of the invention), which is caused or induced e.g. by an additional optical system (e.g. refractive glasses, contact lens, phoropter, and/or other lenses) arranged in front of the at least one eye of the test person. If there is no intrinsic blur of the at least one eye of the test person, the “optically induced blur” can e.g. only include or be an extrinsically induced blur of an otherwise emmetropic eye. If no further optical system is arranged in front of the at least one eye of the test person, the “optically induced blur” can e.g. only include or be the intrinsic blur of the at least one eye of the test person. However, the “optically induced blur” can also be a combination (e.g. a sum) of an intrinsically induced blur and an extrinsically induced blur. It is noted that the “optically induced blur” is generally not causally dependent on the “intrinsically induced blur” or an “extrinsically induced blur”.

If the test images (or the visual object) are used to determine the vision defect of the at least one eye of the test person, it can be intended that the test persons looks at the test images without an additional optical system (i.e. without glasses, contact lenses, phoropter, etc.). In this case, the “optically induced blur” corresponds to the intrinsic blur of the at least one eye of the test person, i.e. the “intrinsically induced blur”. In another application, in which e.g. the “sensitivity to optically induced blur” is to be measured based on the full correction, it can be intended, however, that the test person looks at a test image through an additional optical system (e.g. glasses, contact lenses, phoropter, etc.). In this case, the “optically induced blur” is a combination of any intrinsically induced blur and an extrinsically induced blur. The additional optical system can be designed in such a way that the vision defect of the at least one eye of the test person is only partially corrected. If the correction is only partial, so-called fogging can be caused.

Each filter (or each filter function) is assigned to a specific optically induced blur. In particular, each filter (or each filter function) is defined depending on an optically induced blur. Each original image can thus have one or more associated filters or associated filter functions. Accordingly, each original image can have one or more associated filtered images.

In particular, a filter or a filter function is specific to a predetermined optically induced blur in so far as a viewer (test person) whose at least one eye has a given optically induced blur in combination with the power of an optical system (e.g. refractive glasses) can only just correctly assign an original image filtered with the filter function assigned to this optically induced blur. The expression “only just correctly assign” can be understood or defined as the threshold of a psychometric function, for example, wherein the recognizability of the original image assigned to the filtered image or the probability of the filtered image being correctly assigned to the original image changes significantly when the power of the optical system arranged in front of the at least one eye of the test person changes. This can be determined experimentally, for example, by using psychometric methods to determine the probability of the filtered image being correctly assigned to the original image as a function of the optically induced blur for one test person or for a plurality of test persons.

Application of a filter that is assigned to an optically induced blur or that is specific to an optically induced blur to an original image can ensure recognizability of the original image in the filtered image generated by applying the filter when the filtered image is viewed through the optically induced blur. A filter or a filter function can e.g. correspond to a simple convolution with a Gaussian kernel (example of a low-pass filter) or the application of other filters, such as high-pass or band-pass filters.

In order to assign a filter function or an original image filtered with it to an optically induced blur, the filter function can also be calculated from the predetermined optically induced blur and the eye biometrics. For example, the spatial frequency edge of a low-pass filter can be chosen so that it is proportional to the spatial frequency edge of the momentum transfer function in the presence of optically induced blur (with a proportionality factor of 1, for example, both edges would be identical). The latter can also be approximately calculated as the reciprocal of the viewing angle of the scattering disk for a given optically induced blur. The viewing angles can be calculated as described in WO 2019 034525 A1, for example.

For example, each filtered image that belongs to the test image can represent a different original image. However, it is also possible that the test image comprises several filtered images of the same original image, each of these filtered images then being assigned to a different optically induced blur.

### SUMMARY

In the context of the present invention, viewing an object with an “optically induced blur” means in particular that a test person with an optically induced blur can only recognize or perceive the object being viewed as blurred or only to a certain degree. The “optically induced blur” can be described mathematically e.g. using the power vector P commonly used in spectacle optics. The power vector comprises the three components M, J₀ and J₄₅, where M designates the spherical equivalent and J₀, J₄₅ designate the astigmatic components. A power vector of P=(0, 0, 0) therefore means that an object is viewed without optical blur, whereas a power vector of P=(M,0, 0) with M≠0 e.g. means that the object is viewed with a specific optical blur, which in this case is quantified by M. As already mentioned, the “optically induced blur” can be specified or quantified e.g. using a power vector. In this way, a corresponding power vector can be additionally or alternatively assigned to a filter or filter function used in the context of the present invention, which is specific to a given optically induced blur. In addition to the deviation of the correction arranged in front of the eye (e.g. expressed as a power vector), the optically induced blur can also depend on the pupil size. Both a power vector and a pupil size can therefore be used to quantify the optically induced blur.

Corresponding filters can be used as a convolution of a point spread function, in particular of an uncorrected or not fully corrected eye, with the original image to simulate an image with optically induced blur, as is described e.g. in D. Kordek, L. K. Young and J. Kremláček: “Comparison between optical and digital blur using near visual acuity”, Scientific Reports 11, 3437 (2021), DOI 10.1038/s41598-021-82965-z. The quantification of an optically induced blur using only a power vector without explicitly specifying a pupil size can be done e.g. by assuming an average pupil size (e.g. 1.5 mm radius), and/or using a pupil calculated or predicted from other parameters (cf. WO 2013 087212 A1), and/or—particularly preferably—using an individually measured pupil.

Within the scope of the present invention, it has been found that it is helpful to use test images that are designed in such a way that, with different optically induced blur, they each represent different objects that are easily recognizable by the test person and that can be easily and reliably named. With the help of the present invention, it is e.g. possible for the person being examined or the test person to recognize an object A without optically induced blur (power vector P=(0, 0, 0)), but an object B in the case of an optically induced blur not equal to zero (e.g. power vector P=(M, 0, 0)). With the visual objects or test images according to the invention, it is particularly possible for the test person to recognize a specific original image (from the several predetermined original images) depending on the optically induced blur, namely the original image that was filtered with the filter specific to the corresponding optically induced blur. This advantageously makes the decision of the tested person for a specific object or original image faster and clearer. With the help of the present invention, the determination of the test person's visual properties can thus be carried out more easily, more reliably and also more quickly.

In a preferred embodiment, each of the filtered images is provided with a filter associated with its represented original image and specific to a predetermined optically induced blur such that in the test image
when viewed with a first predetermined optically induced blur, a first one of the at least two original images is better recognizable than each of the remaining original images, which differs from the first original image (or does not correspond to the second original image), and
when viewed with a second predetermined optically induced blur, a second one of the at least two original images is better recognizable than each of the remaining original images, which differs from the second original image (or does not correspond to the second original image).

“When viewed (viewing the visual object or the test image) with a predetermined optically induced blur” is in particular intended to mean that the viewing takes place with otherwise identical viewing conditions (e.g. same brightness, same distance) and otherwise identical visual properties (e.g. with otherwise ideal visual properties or with otherwise average or standard visual properties). The term “otherwise” refers to all visual properties with the exception of the optically induced blur. In other words, viewing with a first predetermined optically induced blur differs from viewing with a second predetermined optically induced blur only in the different optically induced blur itself, but not in other parameters (in particular parameters of the at least one eye).

In particular, when viewed with the first predetermined optically induced blur, substantially only the first one of the at least two original images is recognizable, whereas the remaining original images (different from the first original image) (e.g. the second original image) can preferably only be perceived as a blurred and/or unclear background. An unclear background may be an image that is perceived with a low or too low contrast. Accordingly, particularly when viewed with the second predetermined optically induced blur, substantially only the second of the at least two original images is recognizable, whereas the remaining original images (different from the second original image) (i.e., e.g., the first original image) can preferably only be perceived as a blurred background.

In particular, a filtered image is substantially only recognizable as the respective associated original image when viewed with a predetermined optically induced blur associated with the original image of the filtered image. In particular, this filtered image is substantially only perceived as a blurred background when viewed with an optically induced blur that deviates from the predetermined optically induced blur associated with the original image of the filtered image, in particular by a certain value.

In a further preferred embodiment, each of the filtered images is provided with a filter associated with its original image and specific to a predetermined optically induced blur such that the filtered image, when viewed with the predetermined specific optically induced blur, has a predetermined similarity and/or a predetermined recognizability, in particular a predetermined minimum similarity and/or a predetermined minimum recognizability, to its associated original image. Preferably, the filtered image, when viewed with the predetermined specific optically induced blur, has a similarity of (at least) 50%, more preferably (at least) 60%, even more preferably (at least) 70%, even more preferably (at least) 80%, even more preferably (at least) 90%, even more preferably (at least) 95% and most preferably (at least) 99%. The values given above can in particular be recognition probabilities in a psychometric test. However, if other similarity measures are used, other values can also be advantageous, which e.g. do not necessarily have to be between 0% and 100%.

In particular, the filter (or the corresponding filter function) associated with a specific original image and specified for a specific optically induced blur is characterized in that the associated original image filtered with this filter (or with this filter function) has a predetermined, in particular minimum, similarity to the associated original image when viewed with the associated or specific optically induced blur.

In a further preferred embodiment, the at least two filtered images comprise a first filtered image F₁(B₁) and a second filtered image F₂(B₂).

The first filtered image F₁(B₁) represents a first original image B₁ filtered with a first filter or a first filter function F₁. The second filtered image F₂(B₂) represents a second original image B₂ filtered with a second filter or a second filter function F₂, which differs from the first original image B₁. In particular, a similarity S₁₁₁ of the first filtered image F₁(B₁) to its original image B₁ when viewed with a first optically induced blur P₁ is greater than a similarity S₂₁₂ of the second filtered image F₂(B₂) to its original image B₂ when viewed with the first optically induced blur P₁. It therefore applies: S₁₁₁>S₂₁₂. In general, S_{abc} denotes the similarity of the a-th original image Bₐ filtered with the a-th filter function Fₐ, i.e. of Fₐ(Bₐ), to the c-th original image B_{c} when viewed with the b-th optically induced blur P_{b}. If the optically induced blur is understood as a function, it can be written

$${S}_{abc} = S ( {P}_{b} ( {F}_{a} ( {B}_{a} ) ) , {B}_{c} ) ,$$

where S(.,.) denotes any similarity function of two images, and P_{b}(.) represents the effect of the optically induced blur P_{b} on an image.

In particular, it applies for i∈ [symbol] ⁺; k∈ [symbol] ⁺; i≤N; i≤M; k≤N; and i≠k:

Sᵢᵢᵢ>Sₖᵢₖ,
where Sᵢᵢᵢ is a similarity of an i-th filtered image Fᵢ(Bᵢ) to its original image Bᵢ when viewed with an i-th optically induced blur Pᵢ, and where Sₖᵢₖ is a similarity of a k-th filtered image Fₖ(Bₖ) to its original image Bₖ when viewed with the i-th optically induced blur Pᵢ. Here, Fᵢ(Bᵢ) denotes the i-th original image Bᵢ filtered with an i-th filter function Fᵢ. Similarly, Fₖ(Bₖ) denotes the k-th original image Bₖ filtered with a k-th filter function Fₖ. Furthermore, N denotes the number of original images used and M the number of predetermined different optically induced blurs. N does not necessarily have to match with M.

The similarity of two images can be determined or defined e.g. using image metrics to determine differences between the two images. Known methods or metrics such as “cross-entropy”, “Kullback-Leibler divergence”, “Earth-mover's metric”, etc. can be used for this purpose. Further examples of image metrics that can also be used in the context of the present invention to define the similarity of two images are listed or described e.g. in U.S. Pat. No. 6,493,023B1. The similarity can be defined e.g. as the negative or reciprocal of the image metric representing a difference.

Alternatively or additionally, the similarity can be determined or defined using a physiologically motivated model. This can be done e.g. using a so-called “spatial standard observer”, which is described in the patent documents WO2006/079115A2 and U.S. Pat. No. 7,783,130B2. In particular, these documents describe the determination of an image metric (“visibility metric”) as can be used to define the similarity of two images in the context of the present invention. Further information on physiologically motivated models and/or image metrics that can be used in the context of the present invention can be found e.g. in the following publications:
A. B. Watson et al: “A standard model for foveal detection of spatial contrast”, Journal of Vision (2005) 5, Seiten 717-740, DOI: 10.1167/5.9.6;
A. B. Watson et al: “Predicting visual acuity from wavefront aberrations”, Journal of Vision (2008) 8 (4): 17, Seiten 1-19, DOI: 10.1167/8.4.17;
A. B. Watson et al: “Blur clarified: A review and synthesis of blur discrimination”, Journal of Vision (2011) 11 (5): 10, Seiten 1-23, DOI: 10.1167/11.5.10;
A. B. Watson et al: “Modeling acuity for optotypes varying in complexity”, Journal of Vision (2012) 12 (10): 19, Seiten 1-19, DOI: 10.11167/12.10.19.

The disclosure of all of the above-mentioned patents and publications is hereby expressly referred to. Since the determination or definition of image metrics as such is familiar to the person skilled in the art, this will not be discussed further in the context of the present invention. A specific embodiment for the definition or determination of a similarity or a similarity function will be described below in connection with exemplary embodiments of the present invention.

Alternatively or additionally, the similarity of two images can also be determined or defined on the basis of a recognizability, with the recognizability bing determined by a model based on data from a test with test persons.

Alternatively or additionally, the similarity of two images can be quantified by calculating the classification probability of a classifier (e.g. neural network or other machine learning model), wherein the classifier was previously trained with an image data set that contained at least the original images. A specific embodiment of a classifier will be described below in connection with exemplary embodiments of the present invention. However, training must not be carried out with blurred images.

In a further preferred embodiment, it applies (in particular, the filters or filter functions are selected such that):

S₁₁₁>S₁₂₁; and/or

S₂₂₂>S₁₂₁; and/or

S₂₂₂>S₂₁₂;
where S₁₁₁ is the similarity of the first filtered image F₁(B₁) to its original image B₁ when viewed with the first blur P₁,
where S₂₂₂ is the similarity of the second filtered image F₂(B₂) to its original image B₂ when viewed with a second blur P₂,
where S₁₂₁ is the similarity of the first filtered image F₁(B₁) to its original image B₁ when viewed with the second blur P₂, and
where S₂₁₂ is the similarity of the second filtered image F₂(B₂) to its original image B₂ when viewed with the first blur P₁.

The relationship S₁₁₁>S₁₂₁ means in words that the similarity S₁₁₁ of the first filtered image F₁(B₁) to its original image B₁ when viewed with the first blur P₁ is greater than the similarity S₁₂₁ of the first filtered image F₁(B₁) to its original image B₁ when viewed with a second blur P₂.

In particular, it applies for i∈ [symbol] ⁺; k∈ [symbol] ⁺; i≤N; i≤M; k≤N; k≤M; and i≠k:

Sᵢᵢᵢ>Sᵢₖᵢ,
where Sᵢᵢᵢ is a similarity of an i-th filtered image Fᵢ(Bᵢ) to its original image Bᵢ when viewed with an i-th blur Pᵢ, and where Sᵢₖᵢ is the similarity of an i-th filtered image Fᵢ(Bᵢ) to its original image Bᵢ when viewed with a k-th blur Pₖ. Here, Fᵢ(Bᵢ) denotes the i-th original image Bᵢ filtered with an i-th filter function Fᵢ. Furthermore, N denotes the number of predetermined original images and M denotes the number of predetermined different optically induced blurs. N does not necessarily have to match with M.

The relationship S₂₂₂>S₁₂₁ means in words that a similarity S₂₂₂ of the second filtered image F₂(B₂) to its original image B₂ when viewed with a second blur P₂ is greater than a similarity S₁₂₁ of the first filtered image F₁(B₁) to its original image B₁ when viewed with a second blur P₂.

In particular, it applies for i∈ [symbol] ⁺; k∈ [symbol] ⁺; i≤N; k≤N; k≤M and i≠k:

Sₖₖₖ>Sᵢₖᵢ,
where Sₖₖₖ is the similarity of the k-th filtered image Fₖ(Bₖ) to the associated k-th original image Bₖ when viewed with a k-th blur Pₖ, and where Sᵢₖᵢ is the similarity of the i-th filtered image Fᵢ(Bᵢ) to the associated i-th original image Bᵢ when viewed with a k-th blur Pₖ. Here, N denotes the number of predetermined original images and M denotes the number of predetermined different optically induced blurs. N does not necessarily have to match with M.

The relationship S₂₂₂>S₂₁₂ means in words that a similarity S₂₂₂ of the second filtered image F₂(B₂) to its original image B₂ when viewed with a second blur P₂ is greater than a similarity S₂₁₂ of the second filtered image F₂(B₂) to its original image B₂ when viewed with the first blur P₁.

In particular, it applies for i∈ [symbol] ⁺; k∈ [symbol] ⁺; i≤M; k≤N; k≤M; and i≠k:

Sₖₖₖ>Sₖᵢₖ,
where Sₖₖₖ is the similarity of the k-th filtered image Fₖ(Bₖ) to the associated k-th original image Bₖ when viewed with a k-th blur Pₖ, and where Sₖᵢₖ is the similarity of the k-th filtered image Fₖ(Bₖ) to the k-th original image Bₖ when viewed with the i-th blur Pᵢ. Here, N denotes the number of predetermined original images and M denotes the number of predetermined different optically induced blurs. N does not necessarily have to match with M.

In a further preferred embodiment it applies (in particular the filters or filter functions are chosen such that):

S₁₁₂<S₁₁₁ and S₁₁₂<S₂₂₂; and/or

S₁₂₂<S₁₁₁ and S₁₂₂<S₂₂₂;
where S₁₁₁ is the similarity of the first filtered image F₁(B₁) to its original image B₁ when viewed with the first blur P₁
where S₂₂₂ is the similarity of the second filtered image F₂(B₂) to its original image B₂ when viewed with a second blur P₂,
where S₁₁₂ is the similarity of the first filtered image F₁(B₁) to the original image B₂ when viewed with the first blur P₁ and
where S₁₂₂ is the similarity of the first filtered image F₁(B₁) to the original image B₂ when viewed with the second blur P₂.

The relationships S₁₁₂<S₁₁₁ and S₁₁₂<S₂₂₂ mean in words that a similarity S₁₁₂ of the first filtered image F₁(B₁) to the second original image B₂ when viewed with the first blur Pᵢ is both smaller than the similarity S₁₁₁ of the first filtered image F₁(B₁) to its original image B₁ when viewed with the first blur P₁ and smaller than a similarity S₂₂₂ of the second filtered image F₂(B₂) to its original image B₂ when viewed with the second blur P₂.

In particular, it applies for i∈ [symbol] ⁺; k∈ [symbol] ⁺; i≤N; i≤M; k≤N; k≤M; and i≠k:

Sᵢᵢₖ<Sᵢᵢᵢ and Sᵢᵢₖ<Sₖₖₖ,
where Sᵢᵢᵢ is the similarity of the i-th filtered image Fᵢ(Bᵢ) to the corresponding i-th original image Bᵢ when viewed with an i-th blur Pᵢ, where Sₖₖₖ is the similarity of the k-th filtered image Fₖ(Bₖ) to the corresponding k-th original image Bₖ when viewed with a k-th blur Pₖ, and where Sᵢᵢₖ is the similarity of the i-th filtered image Fᵢ(Bᵢ) to the k-th original image Bₖ when viewed with an i-th blur Pᵢ. Here, N denotes the number of predetermined original images and M denotes the number of predetermined different optically induced blurs. N does not necessarily have to match with M.

The relationships S₁₂₂<S₁₁₁ and S₁₂₂<S₂₂₂ mean in words that the similarity S₁₂₂ of the first filtered image F₁(B₁) to the second original image B₂ when viewed with a second blur P₂ is both smaller than the similarity S₁₁₁ of the first filtered image F₁(B₁) to its original image B₁ when viewed with the first blur P₁ and smaller than the similarity S₂₂₂ of the second filtered image F₂(B₂) to its original image B₂ when viewed with the second blur P₂.

In particular, it applies for i∈ [symbol] ⁺; k∈ [symbol] ⁺; i≤N; i≤M; k≤N; k≤M; and i≠k:

Sᵢₖₖ<Sᵢᵢᵢ and Sᵢₖₖ<Sₖₖₖ,
where Sᵢᵢᵢ is the similarity of the i-th filtered image Fᵢ(Bᵢ) to the associated i-th original image Bᵢ when viewed with an i-th blur Pᵢ, where Sₖₖₖ is the similarity of the k-th filtered image Fₖ(Bₖ) to the associated k-th original image Bₖ when viewed with a k-th blur Pₖ, and where Sᵢₖₖ is the similarity of the i-th filtered image Fᵢ(Bᵢ) to the k-th original image Bₖ when viewed with a k-th blur Pₖ. N denotes the number of predetermined original images and M denotes the number of predetermined different optically induced blurs. N does not necessarily have to match with M.

In a further preferred embodiment, the at least two filtered images are arranged next to one another and/or in an overlapping fashion, in particular at least partially lying on top of one another. In particular, the filtered images of the test image can be arranged next to each other, e.g. in a row, a column, a matrix, or in an otherwise non-overlapping fashion. Alternatively or additionally, the filtered images of the test image can be arranged in an overlapping fashion and in particular one above the other. In the latter case, the overlapping images Fⱼ(Bⱼ) with j≠i, which do not appear sharp with blur Pᵢ, create a (blurred) background from which the easily recognizable image Fᵢ(Bᵢ) stands out with the original image Bᵢ, so that a decision for classifying the visual impression as image Bᵢ of the person viewing or the test person is easy. At least some of the filtered images or all of the filtered images can be superimposed (e.g. added). The test image generated e.g. by superimposing the individual filtered images can then be displayed or presented to the test person using common means (e.g. the test image can be displayed on a screen or be printed).

In a further preferred embodiment, the filters or filter functions are adapted to average higher-order aberrations of an eye. Alternatively, the filters or filter functions are adapted to individual higher-order aberrations of at least one eye of the test person. In this way, it can advantageously be achieved that the test person recognizes the original image specifically filtered for its optically induced blur better, i.e. more clearly and more quickly. In other words, the recognizability can be improved individually or at least on average.

Any aberrations can be taken into account e.g. using a prefiltering technique. With the prefiltering technique, the original image is convolved with a function that has small values where the point spread function (in particular of an uncorrected or not fully corrected eye) has large values and vice versa, e.g. with the reciprocal of the point spread function or with a Wiener filter based on the point spread function. Negative pixel intensities of an image thus prefiltered can be brought to a displayable range (e.g. relative intensities between 0 and 1) by normalization. If an image thus prefiltered is viewed through an optically induced blur, the viewer can perceive the (sharp) original image, which, however, has a lower contrast due to the normalization. Further information on this can be found e.g. in Fu-Chung Huang: “A Computational Light Field Display for Correcting Visual Aberrations”, EECS Department, University of California, Berkeley, 2013, Technical Report No. UCB/EECS-2013-206, http://www.eecs.berkeley.edu/Pubs/TechRpts/2013/EECS-2013-206.html. In particular, higher-order aberrations can now be taken into account by taking into account the phase error of higher (Zernike)-order aberrations in addition to the second (Zernike)-order phase error when calculating the point spread function from the phase error in the entrance pupil (often called the “wavefront”). The calculation of the point spread function from the phase error in the entrance pupil is described e.g. in D. Kordek, L. K. Young and J. Kremláček: “Comparison between optical and digital blur using near visual acuity”, Scientific Reports 11, 3437 (2021), DOI 10.1038/s41598-021-82965-z.

Another independent aspect for solving the object relates to a method for determining one or more visual properties of a test person, comprising:
providing a visual object according to the invention, wherein providing the visual object in particular comprises presenting of the visual object. The presentation can in particular be carried out using common means including a projection onto the retina of a test person, or by displaying on a screen or by printing, e.g. on paper. In particular, the visual object can be presented to the test person.

In a preferred embodiment, providing a visual object comprises the following steps:
providing at least two original images;
generating at least two filtered recognition images by applying filters (or filter functions) specific to a predetermined optically induced blur to the at least two original images such that when the filtered recognition images are viewed with the predetermined optically induced blur, each filtered recognition image has a predetermined similarity, in particular a predetermined minimum similarity, to the respective original image associated with the filtered recognition image.

A “filtered recognition image” is in particular a filtered original image that is filtered such that the test person can recognize it as the original image when viewed with an optically induced blur.

Preferably, the method for providing the visual object according to the invention comprises the steps of:
adapting the filter function(s) to average higher-order aberrations of an eye; and/or
adapting the filter function(s) to individual higher-order aberrations of at least one eye of the test person.

In a further preferred embodiment, providing the visual object comprises determining the filter (or a corresponding filter function) specific to a predetermined optically induced blur for each original image. Determining the filter specific to a predetermined optically induced blur for each original image comprises in particular calculating an optical transfer function associated with the respective optically induced blur. For example, the filtering can correspond to at least a partial phase reversal and/or phase rectification. Methods for this can be found e.g. in the publication by S. Ravikumar et al: “Phase changes induced by optical aberrations degrade letter and face acuity”, Journal of Vision (2010) 10 (14): 18, pages 1-12, DOI: 10.1167/10.14.18, the disclosure of which is hereby expressly incorporated by reference. To this end, it is necessary to calculate the optical transfer function, i.e. the Fourier transform of the point spread function for the respective optically induced blur Pᵢ.

Other filters can also be used. For example, to generate a filtered image, at least two filters from the filter categories low-pass filter, band-pass filter, and high-pass filter can be used. When using low-pass, high-pass and/or band-pass filters, the characteristic frequencies of the filters can be selected depending on the characteristic frequencies of the optical transfer function generated by an optically induced blur. For example, in the case of a combination of a low-pass or band-pass filter applied to a first original image with a high-pass filter applied to a second original image, the filter edge of the low-pass filter (or the high-frequency filter edge of the band-pass filter) can be selected to be lower than the spatial frequency of the filter edge of the optical transfer function generated by a predetermined optically induced blur, and the filter edge of the high-pass filter can accordingly be selected to be higher than the frequency of the filter edge of the optical transfer function.

What is also possible is an optimization process in which filter parameters are adjusted (e.g. the above-mentioned characteristic frequencies of the filters, or the filter parameters used in the “prefiltering technique”). An optimization function G_{θ}(.) to be maximized can be determined e.g. from the similarities Sᵢⱼₖ and depends implicitly on the parameters θᵢ of the i-th filter functions Fᵢ(.):

$${G}_{θ} = G ( {θ}_{1} , {θ}_{2} , … , {θ}_{N} ) = {\sum }_{j = 1}^{M} {\sum }_{i , k = 1}^{N} {w}_{ijk} ( 2 {δ}_{ik} {δ}_{ij} - 1 ) {S}_{ijk} ( {θ}_{i} ) .$$

The maximum of G_{θ} is thus formed via the parameters θ of the filter functions:

$${θ}^{*} = arg {max}_{θ} ( {G}_{θ} ) .$$

It should be noted that several filter functions can be identical. For example, there may be only two types of filter functions, e.g. a high-pass filter and a band-pass filter, the parameters of which are optimized. In this case (as in the embodiment), the filter functions F₁(θ₁)=F₃(θ₃)=F₂ᵢ₋₁(θ₂ᵢ₋₁)= . . . =F_{N-1}(θ_{N-1})=F_{BP}(θ_{BP}) would correspond to one and the same band-pass filter, and the filter functions F₂(θ₂)=F₄(θ₄)=F₂ᵢ(θ₂ᵢ)= . . . =F_{N}(θ_{N})=F_{HP}(θ_{HP}) would correspond to one and the same high-pass filter. Three parameters f₁, f₂ and f₃ of the filter functions are varied during optimization using conventional optimization methods to find a maximum of G_{θ}(f₁, f₂, f₃), where θ₂ᵢ₋₁=θ_{BP}=(f₁, f₂) is the lower and upper edge of the band-pass filter, and θ₂ᵢ=θ_{HP}=f₃ is the frequency edge of the high-pass filter. The weights wᵢⱼₖ are positive as usual and can be chosen to be identically 1 in the simplest case, or e.g. as follows:

$${w}_{ijk} = \frac{{δ}_{ik}}{N} + \frac{1 - {δ}_{ik}}{N ( N - 1 )} .$$

In a further preferred embodiment, providing the visual object comprises the following steps:
filtering a plurality N of (predetermined) different original images Bᵢ with 1≤i≤N, for a plurality M of (predetermined) optically induced blurs Pⱼ with 1≤j≤M, where M and N are each a positive integer with M<N, and
selecting those filtered images Fⱼ(Bᵢ) the similarity Sᵢᵢ of which to the original images Bᵢ exceeds a first threshold value when viewed with an optically induced blur Pᵢ (i.e. is as high as possible), and the similarity Sᵢⱼ of which to the other original images Bⱼ with j≠i when viewed with an optically induced blur Pᵢ falls below a second threshold value (i.e. is as low as possible). This can be understood as an optimization across all subsets with M images. In particular, the first threshold value is greater than or equal to the second threshold value. Preferably, only those M images are selected from a plurality of N original images that are particularly suitable as images within the scope of the invention (due to the selection, M<N). The reason for the selection is that the recognizability of the original images in the filtered images does not only depend on the filter functions used, but also on the distribution of the information required for assignment across different spatial frequencies. For example, a first original image that can already be easily recognized based on the low-frequency components can be exceptionally well-suited to being filtered with a low-pass filter in order to later be combined with a high-pass filtered second image. However, if the filters are swapped and the first original image is treated with a high-pass filter and the second original image is treated with a low-pass filter, it may be that the high-frequency image components of the first original image are no longer (or no longer as well-) suited for recognizing the original image in the filtered image because e.g. they are too unspecific or their amplitudes were already too low in the first original image.

In a further preferred embodiment, the selection of filtered images represents an optimization across all subsets with R original images. The optimization is preferably carried out by maximizing a target function, wherein the target function is defined in particular as follows:

Σᵢⱼₖwᵢⱼₖ(2δᵢⱼδᵢₖ−1)Sᵢⱼₖ, where
δ represents the Kronecker symbol and wᵢⱼₖ are weights with wᵢⱼₖ≥0 and Σᵢⱼₖ wᵢⱼₖ>0. In particular, optimization is only carried out across subsets of the original images β with a constant number of elements, wherein the filter functions are now fixedly predetermined. An optimization function G_{β} to be maximized can be determined e.g. from the similarities Sᵢⱼₖ and depends implicitly on the parameters θᵢ of the i-th filter functions Fᵢ(.):

$${G}_{β} = {\sum }_{j = 1}^{M} {\sum }_{\begin{matrix}i , k = 1 , \\ i , k ∈ β\end{matrix}}^{N} {w}_{ijk} ( 2 {δ}_{ik} {δ}_{ij} - 1 ) {S}_{ijk} ( {θ}_{i} ) .$$

The most suitable set of images can be determined by

$${β}^{*} = arg {max}_{β ∈ T} ( {G}_{β} )$$

where T is the set of all subsets of the indices of the original images with R elements.

The weights wᵢⱼₖ can all be the same (e.g. wᵢⱼₖ=1 for all i, j, k). However, it is advantageous to scale the weights with the similarities of the diagonal elements as follows:

$${w}_{ijk} = \frac{1}{\sqrt{{S}_{iii} ( {θ}_{i} ) \cdot {S}_{kkk} ( {θ}_{k} )}}$$

for any k.

In a further preferred embodiment, the method comprises the steps of:
evaluating a test person's reaction to the visual object, wherein evaluating in particular comprises
   determining a test person's ametropia, and/or
   determining the test person's sensitivity to an optically induced blur, and/or
   determining a filter (or a filter function) optimal for the test person.

A test person's reaction can be e.g. a response, a motoric action (such as a demonstration, tapping representations on a board, a device or a screen) and/or an eye movement. The determination of a test person's ametropia can thus advantageously be carried out without having to generate an optically induced blur (by means of additional aids).

The determination of a filter optimal for the test person can comprise determining a filtering with a fixed but unknown optically induced blur, in which the test images according to the invention are used, with the aim of finding a filtering that enables the best possible recognition of an image filtered with the filter on a screen.

A further independent aspect for solving the object relates to a computer program product comprising machine-readable program code which, when loaded on a computer, is suitable for executing the method according to the invention described above. In particular, a computer program product is to be understood as a program stored on a data carrier. In particular, the program code is stored on a data carrier. In other words, the computer program product comprises computer-readable instructions which, when loaded into a memory of a computer and executed by the computer, cause the computer to carry out a method according to the invention described above.

In particular, the computer program product can comprise a computer-readable storage medium having code stored thereon, wherein the code, when executed by a processor, causes the processor to implement a method according to the invention. The invention thus provides a computer program product, in particular in the form of a storage medium or a data stream, which contains program code which, when loaded and executed on a computer, is designed to carry out a method for providing a visual object according to the invention and/or for determining one or more visual properties of a test person.

The statements made above or below regarding the embodiments of the first aspect also apply to the above-mentioned further independent aspects and in particular to the related preferred embodiments. In particular, the statements made above and below regarding the embodiments of the other independent aspects also apply to an independent aspect of the present invention and to the related preferred embodiments.

In the following, individual embodiments for solving the object will be described by way of example using the figures. Here, the individual embodiments described partly include features that are not absolutely necessary to carry out the claimed subject matter, but which provide desired properties in certain applications. Thus, embodiments that do not include all the features of the embodiments described below should also be considered to be covered by the technical teaching described. Furthermore, in order to avoid unnecessary repetition, certain features will only be mentioned in relation to individual ones of the embodiments described below. It should be noted that the individual embodiments should therefore not only be considered on their own, but also in combination with one another. Based on this combination, the person skilled in the art will recognize that individual embodiments can also be modified by incorporating individual or several features of other embodiments. It is pointed out that a systematic combination of the individual embodiments with single or several features described with respect to other embodiments may be desirable and useful and should therefore be considered and also regarded as included in the description.

### BRIEF DESCRIPTION OF THE DRAWINGS

FIG. 1a shows an exemplary first original image;

FIG. 1b shows an exemplary second original image;

FIG. 2a shows an exemplary first filtered image, namely the first original image of FIG. 1a filtered with a first filter;

FIG. 2b shows an exemplary second filtered image, namely the second original image of FIG. 1b filtered with a second filter;

FIG. 3 shows a visual object or test image according to a preferred embodiment of the invention, in which the first filtered image of FIG. 2a and the second filtered image of FIG. 2b are superimposed;

FIG. 4 shows: in the top row, six exemplary original images B₁ to B₆; in the middle row, masks for a high-frequency background associated with the respective original images, each of which was generated by applying a low-pass filter to the original images above them; and in the bottom row, the original images of the top row, with a high-frequency background added in the area of the masks (black in the respective middle row above);

FIG. 5 shows: in the top row the six exemplary original images of FIG. 4, which were filtered with a band-pass filter; and in the bottom row, the six exemplary original images of FIG. 4, which were filtered with a high-pass filter;

FIG. 6 shows combined filtered images based on the original images of FIG. 4 and the band-pass and high-pass filtered original images of FIG. 5;

FIG. 7 shows tables with calculated similarities of the original images B₁ to B₆ to the combined images shown in FIG. 6 when viewed without optical blur;

FIG. 8 shows: on the left the Zernike coefficients cₙ^{m} of a wavefront error of the at least one eye of a test person; top right a 1024×1024 pixel image of the point spread function of the uncorrected eye with an entrance pupil radius of 2.52 mm for a wavelength of 550 nm; and bottom right an enlargement of the image above.

FIG. 9 shows superimposed filtered images with different optically induced blurs;

FIG. 10 shows tables with calculated similarities of the original images B to B to the combined images shown in FIG. 6 when viewed with a first optical blur P₁;

FIG. 11 shows tables with calculated similarities of the original images B₁ to B₆ to the combined images shown in FIG. 6 when viewed with a second optical blur P₂;

FIG. 12 shows tables with calculated similarities of the original images B₁ to B₆ to the combined images shown in FIG. 6 when viewed with a third optical blur P₃;

FIG. 13 shows tables with calculated similarities of the original images B₁ to B₆ to the combined images shown in FIG. 6 when viewed with a fourth optical blur P₄;

FIG. 14 shows tables with calculated similarities of the original images B₁ to B₆ to the combined images shown in FIG. 6 when viewed with a fifth optical blur P₅; and

FIG. 15 shows a graphical representation of the similarities from the tables of FIGS. 7 and 10 to 14;

### DETAILED DESCRIPTION

It is noted that the figures shown here represent photographic images, each of which cannot be represented as a black and white line drawing without undesirably distorting their character.

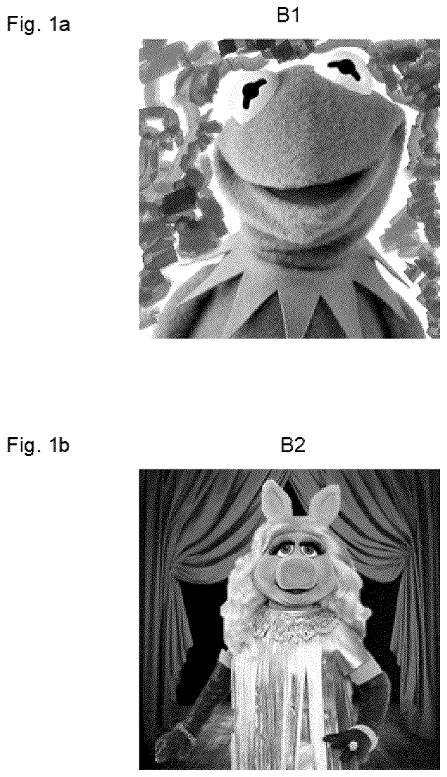

FIG. 1a shows an exemplary first original image. This first original image B₁ shows a photo of “Kermit”, a cartoon character from the well-known “Muppet Show”. FIG. 1b shows an exemplary second original image. This second original image B₂ shows a photo of “Miss Piggy”, another cartoon character from the “Muppet Show”. Both original images or photos are unfiltered and therefore each correspond to an original image.

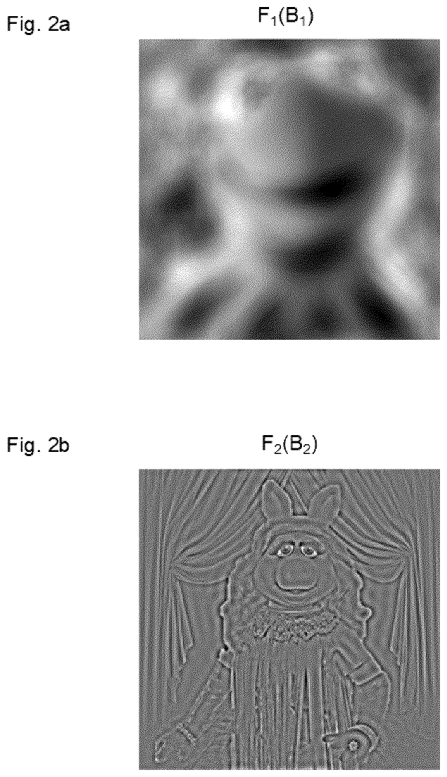

FIG. 2a shows a first filtered image F₁(B₁) provided with a first filter or a first filter function F₁. This first filtered image F₁(B₁) is based on the first original image B₁ or represents the first original image B₁. In particular, the first filtered image F₁(B₁) represents the first original image B₁ to which a first filter or a first filter function F₁ was applied. The first filter F₁ is selected such that the first filtered image Fᵢ(B₁) has a relatively high similarity to the first original image B₁ when viewed with a first optically induced (spherical) blur P₁, which corresponds e.g. to a first power vector P₁=(1.5 dpt, 0, 0). However, if the filtered image F₁(B₁) is viewed with a second optically induced blur P₂ that differs from the first optically induced blur P₁ (in particular by a specific or minimum value), the first filtered image F₁(B₁) appears unclear or blurred and/or has less similarity to the first original image than when viewed with the first optically induced blur, for which the first filter Fᵢ is specific. As can be seen from FIG. 2a, the filtered image F₁(B₁) shown therein appears unclear or blurred when viewed without optically induced (spherical) blur, which corresponds to a second power vector P₂=(0 dpt, 0, 0). The filter F₁ is therefore a filter specific to the first optically induced blur, since when viewed with the first optically induced blur, the original image B₁ can be clearly seen, whereas when viewed with a different optically induced blur (for example with the second optically induced blur P₂), the original image B₁ can be seen less clearly, poorly, or not at all (or only as a background).

FIG. 2b shows a second filtered image F₂(B₂) provided with a second filter or a second filter function F₁. This second filtered image F₂(B₂) is based on the second original image B₂ or represents the second original image B₂. In particular, the second filtered image F₂(B₂) represents the second original image B₂ to which a second filter or a second filter function F₂ was applied. The second filter F₂ is selected such that the second filtered image F₂(B₂) has a relatively high similarity to the second original image B₂ when viewed with the second optically induced blur P₂, which corresponds to the power vector P₂=(0 dpt, 0, 0). However, if the filtered image F₂(B₂) is viewed with a different optically induced blur that differs from the second optically induced blur P₂ (in particular by a specific or minimum value), the filtered image F₂(B₂) appears unclear or blurred and/or has a lower similarity to the second original image B₂ than when viewed with the second optically induced blur P₂, for which the second filter F₂ is specific. As can be seen from FIG. 2b, the filtered image F₂(B₂) shown therein appears to be clearly recognizable when viewed without optically induced (spherical) blur P, which corresponds to the second power vector P₂=(0 dpt, 0, 0). The filter F₂ is therefore a filter specific to the second optically induced blur P₂, since it allows the original image B₂ to be clearly recognized when viewed with the second optically induced blur P₂, whereas when viewed with a different optically induced blur (for example with the first optically induced blur P₁), the original image B₂ is less clearly recognizable, poorly recognizable, or not recognizable at all (or only as a background).

In the embodiment shown, the filter F₁ used is a low-pass filter, i.e. the filtered image F₁(B₁) shown in FIG. 2a is a low-pass filtered image. In contrast, the filter F₂ used in the embodiment shown is a high-pass filter, i.e. the filtered image F₂(B₂) shown in FIG. 2b is a high-pass filtered image.

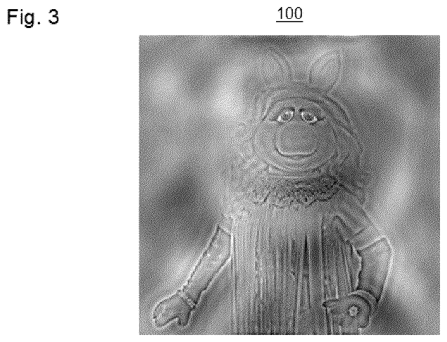

FIG. 3 shows a visual object or a test image 100 according to a preferred embodiment of the invention. In the visual object 100 or in the test image shown, the first filtered image F₁(B₁) shown in FIG. 2a and the second filtered image F₂(B₂) shown in FIG. 2b are combined. In the embodiment shown, the first filtered image F₁(B₁) of FIG. 2a and the second filtered image F₂(B₂) of FIG. 2b are superimposed. In other words, the first filtered image F₁(B₁) and the second filtered image F₂(B₂) completely overlap.

The visual object or test image 100, shown with an edge length corresponding to 8 degrees at a proximity of 0 dpt, can be recognized as “Miss Piggy” (i.e. as the second original image B₂) when viewed with the first spherical blur P₂=(0 dpt, 0, 0). However, if the visual object or test image 100 is viewed with the second spherical blur P₂=(1.5 dpt, 0, 0), it can be recognized as “Kermit” (i.e. as the first original image B₁). The information about which original image a test person recognizes on the test image shown can be used to determine or identify the test person's visual properties (e.g. ametropia). For example, a plurality of visual objects or test images 100 according to the invention can be presented to the test person in order to successively draw conclusions about specific visual properties of the test person based on a corresponding reaction or response from the test person.

Each of the filtered images F₁(B₁) and F₂(B₂) is thus provided with a filter associated with its represented original image B₁ or F₁(B₁) and specific to a predetermined optically induced blur such that on the test image, when viewed with a first predetermined optically induced blur, a first one of the at least two original images (here: “Kermit”) is better recognizable than each of the remaining original images (here: “Miss Piggy”), whereas when viewed with a second predetermined optically induced blur, a second one of the at least two original images (here: “Miss Piggy”) is better recognizable than each of the remaining original images (here: “Kermit”).

Within the scope of the present invention, there is in particular proposed a method for creating a test image from two or more original images containing e.g. symbols, optotypes or other objects that can be easily and clearly recognized. Each image Bᵢ is filtered with a filter function Fᵢ. The filtered image Fᵢ(Bᵢ) has a similarity Sᵢⱼₖ to the original image Bₖ with the optically induced blur Pⱼ. At least a part of the filtered images can then be superimposed (e.g. added) and the image created by the superposition can e.g. be displayed on a screen or be printed. The displayed filtered images can be arranged next to one another (e.g. in a row, a column, a matrix or in an otherwise non-overlapping fashion) and, alternatively or additionally, can also be arranged one above the other. In the latter case, the overlapping images Fⱼ(Bⱼ) with j≠i, which do not appear sharp with the blur Pᵢ, create a background from which the easily recognizable image Fᵢ(Bᵢ) stands out with the original image Bᵢ, so that a decision for classifying the visual impression as image Bᵢ is easy for the person viewing it.

The visual objects or test images according to the invention are designed in particular such that, with different optically induced blur, they each represent different objects that are easily recognizable by the test person and that can be named easily and reliably. For example, the person being examined can recognize an object A without optically induced blur (as a power vector P=(0, 0, 0)), but an object B with an optically induced blur that is different from zero (e.g. power vector P=(M, 0, 0)). With the test images according to the invention, a test person can therefore recognize a respectively different object, in particular depending on the optically induced blur. This means that the person being tested can make a quicker and clearer decision for a particular object.

In order to generate the test images according to the invention, the images Bᵢ can first be filtered in a suitable manner using the filter function Fᵢ in order to create good recognizability with the desired optical blur Pᵢ. The filtering can correspond to at least partial phase reversal and/or phase rectification. To this end, it is necessary to calculate the “optical transfer function” (i.e. the Fourier transform of the “point spread function”) for the respective optically induced blur Pᵢ. Other filters can also be used. For example, at least two filters from the filter categories low-pass filter, band-pass filter, and high-pass filter can be used to generate a filtered image. The filters can also be adapted to the individual or average higher-order aberrations of the eye in order to improve recognizability individually or at least on average.

Particularly good results are achieved by filtering a plurality of different images Bᵢ, with 1≤i≤N, for the optically induced blurs Pⱼ, with 1≤j≤M, where M<N, and then selecting the filtered images Fᵢ(Bᵢ) the similarity Si of which to the original images Bᵢ is as high as possible when viewed with the blur Pᵢ, and, at the same time, the similarity Sᵢⱼ of which to the other images Bⱼ with j≠i when viewed with the blur Pᵢ is as low as possible. This can be understood as an optimization across all subsets with M images, wherein the optimization can be achieved by maximizing a target function.

Possible applications of the invention are in particular:
determining the ametropia without having to generate optically induced blur;
determining the sensitivity to optically induced blur; and/or
determining a filtering in the case of fixed but unknown optically induced blur, in which the test images according to the invention are used, with the aim of finding a filtering that ensures the best possible recognition of an image filtered with the filter on a screen.

When test images are used, as with test images containing optotypes according to the prior art, it is of course important to note that in addition to the optically induced blur, the distance between the test image and the eye of the person being tested plays a major role. It is also important to note that certain optometric parameters (e.g. visual acuity) determined with the test images according to the invention do not necessarily correspond to the same parameters that were determined using test images containing optotypes according to the prior art, and that calibration of the measuring method according to the invention to a standard measuring method might be necessary.

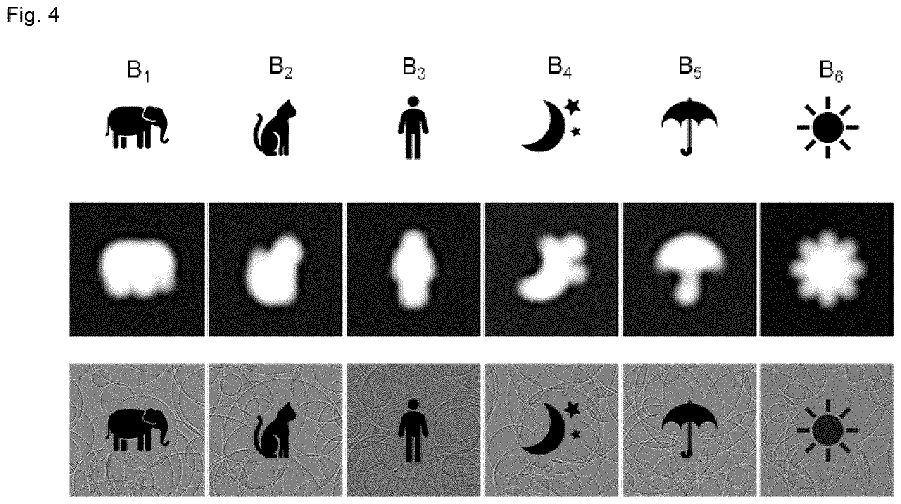

Below, further examples of original images, filtered images, combined or superimposed images and their similarity to the original images will be presented using FIGS. 4 to 14.

FIG. 4 shows six exemplary original images B₁ to B₆ in the top row. Here, original image B₁ represents an elephant, original image B₂ a cat, original image B₃ a person, original image B₄ a moon, original image B₅ an umbrella, and original image B₆ a sun. The middle row of FIG. 4 shows masks corresponding to the original images B₁ to B₆ shown in the row above, which were created by applying a low-pass filter to the original images above them. These masks are used to provide the original images B₁ to B₆ with a high-frequency background. The bottom row of FIG. 4 shows the original images B₁ to B₆ provided with a high-frequency background, with the high-frequency background being added by applying the masks to the original images. In particular, a high-frequency background was added in each image area specified by the masks (shown in black in the masks). The high-frequency background serves in particular to draw the viewer's attention to high-frequency structures in the combined image, provided they can be perceived with a given optically induced blur.

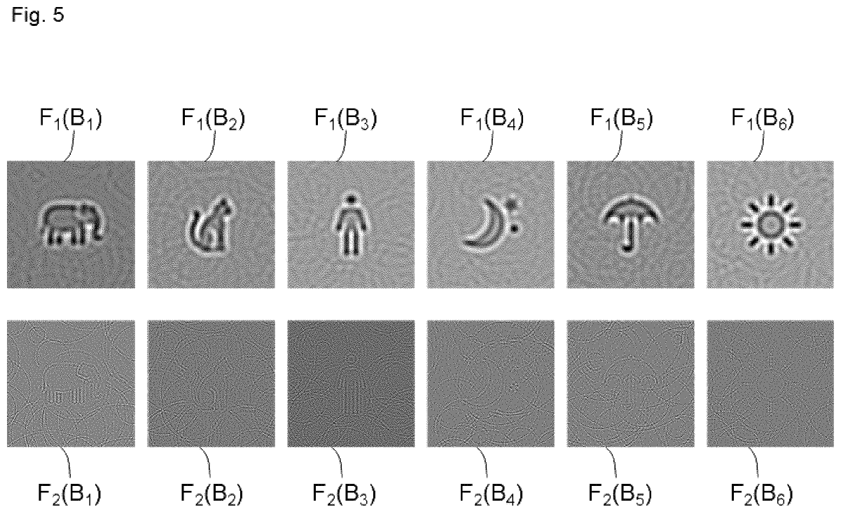

FIG. 5 shows filtered original images, with the original images B₁ to B₆ in the top row of FIG. 5 each having been filtered with a band-pass filter F₁, whereas the original images B₁ to B₆ in the bottom row of FIG. 5 each having been filtered with a high-pass filter F₂. For example, the top left image with the designation F₁(B₁) is the original image B₁ filtered with the band-pass filter F₁. The bottom right image with the designation F₂(B₆) is e.g. the original image B₆ filtered with the high-pass filter F₂.

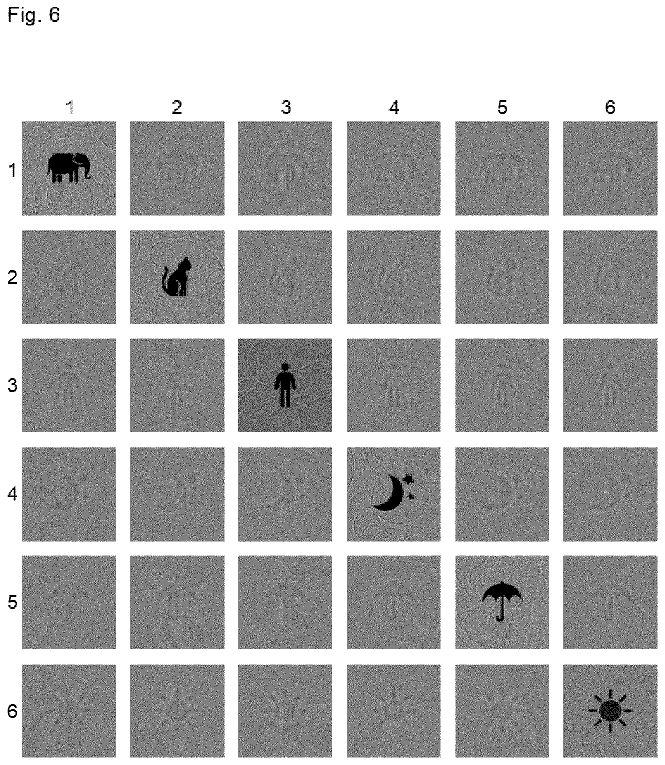

FIG. 6 shows a matrix with combined (or superimposed) filtered images. The images shown in the matrix can be identified by two numbers, the first number indicating the row and the second number indicating the column of the matrix. The images contained in the diagonal of the matrix shown in FIG. 6, i.e. images “11”, “22”, “33”, “44”, “55”, and “66”, correspond to the original images B₁ to B₆. The non-diagonal elements of the matrix contain images that represent a combination or superposition of band-pass and high-pass filtered original images. The same band-pass filtered image was used for the combination in each row, and the same high-pass filtered image in each column. The combination of the band-pass and high-pass filtered original images was carried out by a weighted addition of the filtered images, with the band-pass filtered images being weighted by a factor of 0.2 and the high-pass filtered images being weighted by a factor of 0.8. In this example, the edge length of the individual images is to correspond to 580 arc minutes when viewed.

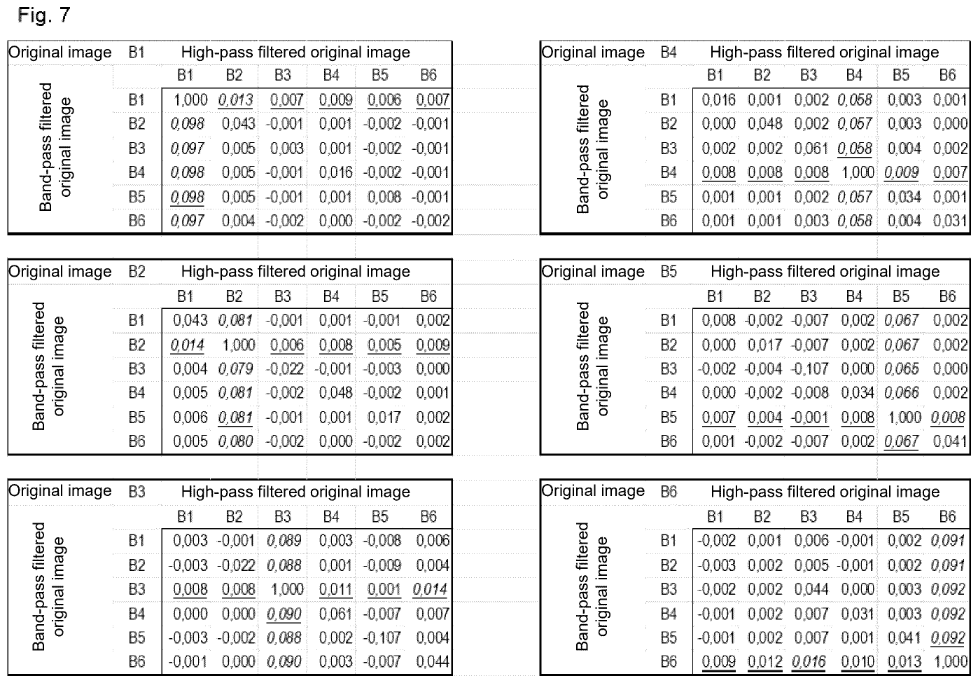

FIG. 7 shows tables with calculated similarities of the original images B₁ to B₆ to the combined images shown in FIG. 6 when viewed without optical blur. For example, the upper left table of FIG. 7 shows the thus calculated values for the similarity of the combined images shown in FIG. 6 to the original image B₁. Accordingly, the lower right table of FIG. 7 shows the calculated values for the similarity of the combined images shown in FIG. 6 to the original image B₆. For example, as can be seen from the upper left table of FIG. 7, the calculated similarity of image “12” (row 1, column 2) to the original image B₁ is 0.013, while the calculated similarity of image “21” (row 2, column 1) to the original image B₁ is 0.098. As expected, the calculated similarity of image “11” (row 1, column 1) to the original image B₁ is 1,000. The values in the other tables in FIG. 7 are to be read accordingly. The highest values in a row or column are shown in italics or underlined in the tables. The calculation of the similarities indicated in the tables in FIG. 7 was carried out using a correlation-based similarity function as an example. Here, the similarity function was respectively applied to an original image and a combined filtered image in order to quantitatively determine the similarity of these two images. An embodiment for the definition or determination of a similarity using a similarity function will be described below.

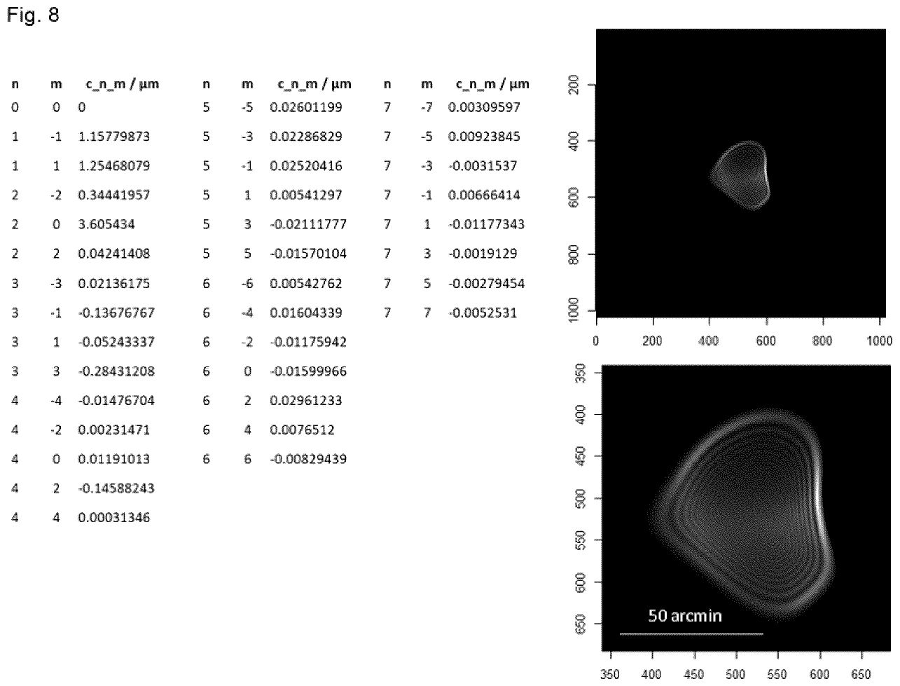

On the left side of FIG. 8, the Zernike coefficients cit of an exemplary wavefront error of the at least one eye of a test person are indicated for the purposes of the examples described below. Furthermore, on the right side of FIG. 8 at the top, a 1024×1024 pixel image of the point spread function of the uncorrected eye with an entrance pupil radius of 2.52 mm for a wavelength of 550 nm is shown. An enlargement of this image is shown immediately below the image. The intensity of the point spread function has been normalized for better representation. For simulation purposes, all orders with n≤1 can also be set to zero. In the example shown, the vision defect of the eye is M=−3.94 dpt, J₀=−0.03 dpt, and J₄₅=−0.27 dpt. The point spread function is shown from the perspective of the person with the aforementioned wavefront errors and corresponds to the visual impression of a point light source.

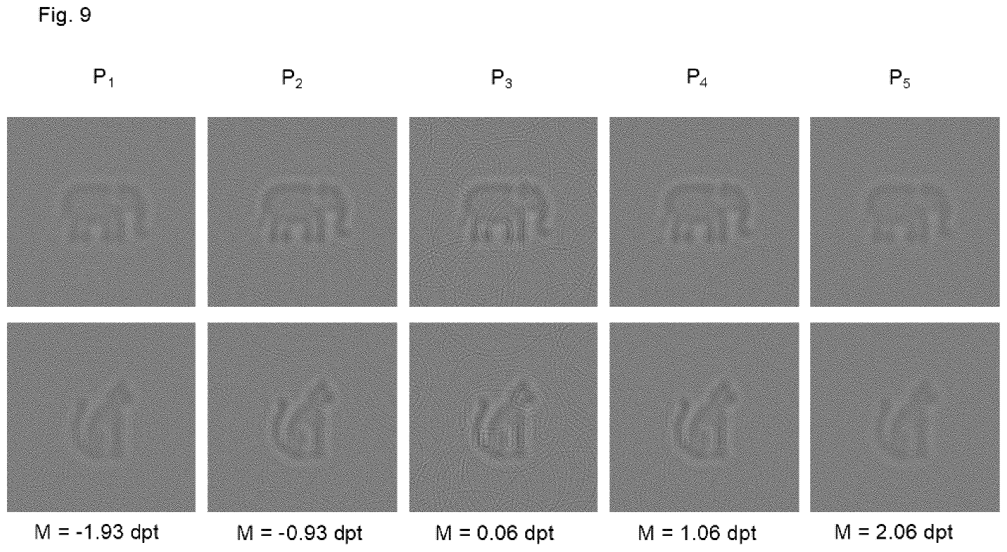

FIG. 9 shows superimposed filtered images at different optically induced blurs. In each of the five columns of FIG. 9, two superimposed filtered images are shown, each viewed at a specific optically induced blur Pᵢ (with i=1, 2, 3, 4, 5). The M component is as indicated under the respective images. The examples shown are based on an eye with the Zernike coefficients indicated in FIG. 8. This eye with an exemplary pupil radius of 2.52 mm has a vision defect of M=−3.94 dpt, J₀=−0.03 dpt, and J₄₅=−0.27 dpt. The higher order aberrations are as indicated in the table in FIG. 8. If this vision defect is corrected with an eye correction that has a power of M₁=−6 dpt, M₂=−5 dpt, M₃=−4 dpt, M₄=−3 dpt, and M₅=−2 dpt with astigmatic components of J₀=0 dpt, and J₄₅=−0.25 dpt at a corneal vertex distance of −3.5 mm (position of the entrance pupil), the optically induced blurs P₁ to P₅ result with the M values indicated in FIG. 9. One of the two superimposed filtered images in FIG. 9 is based on the original image B₁ (“elephant”), and the other of the two superimposed filtered images in FIG. 9 is based on the original image B₂ (“cat”). Here, the images in the top row of FIG. 9 are each a superposition of the images F₁(B₁) and F₂(B₂) of FIG. 5, while the images in the bottom row of FIG. 9 are each a superposition of the images F₂(B₁) and F₁(B₂) of FIG. 5. As can be seen from FIG. 9, depending on which filters the superimposed filtered image is provided with and the optical blur with which the superimposed image is viewed, the superimposed image is more similar to the original image B₁ (“elephant”) or the original image B₂ (“cat”). All images and calculated values of similarities based on the “new symbols” (cat, elephant, etc.) refer to an observation in which the side length of the images corresponds to 580 arc minutes of visual angle.

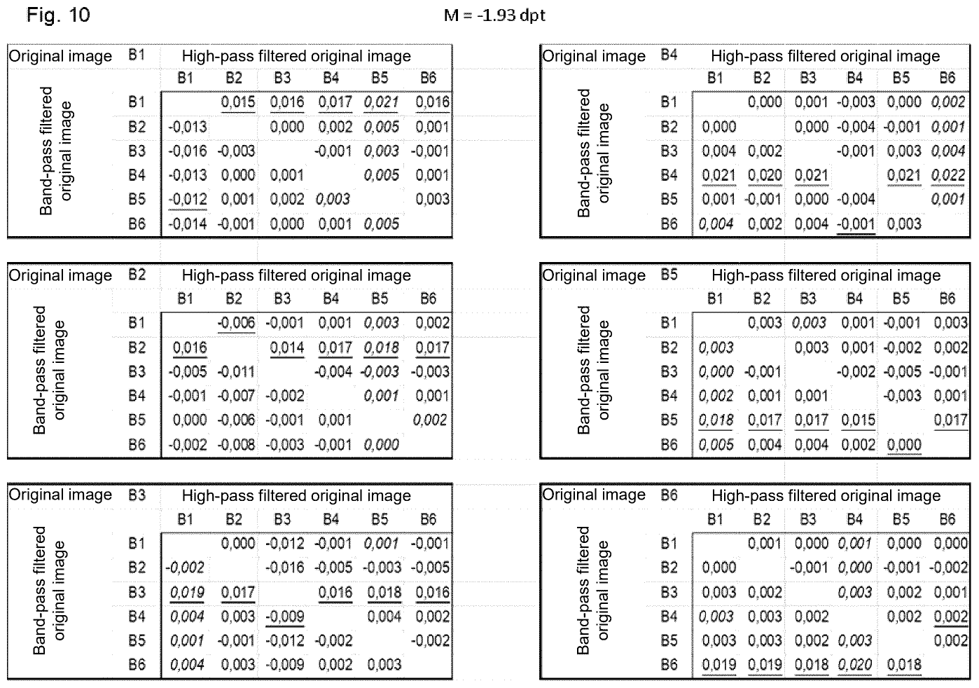

FIG. 10 shows, similar to FIG. 7, tables with calculated similarities of the original images B₁ to B₆ to the combined images shown in FIG. 6. Unlike the tables in FIG. 7, however, the similarity values contained in the tables in FIG. 10 refer to an observation with the optical blur P₁ (M=−1.93 dpt, J₀=−0.03 dpt, J₄₅=−0.02 dpt). The highest values in a row or column are also shown in italics or underlined in the tables in FIG. 10.

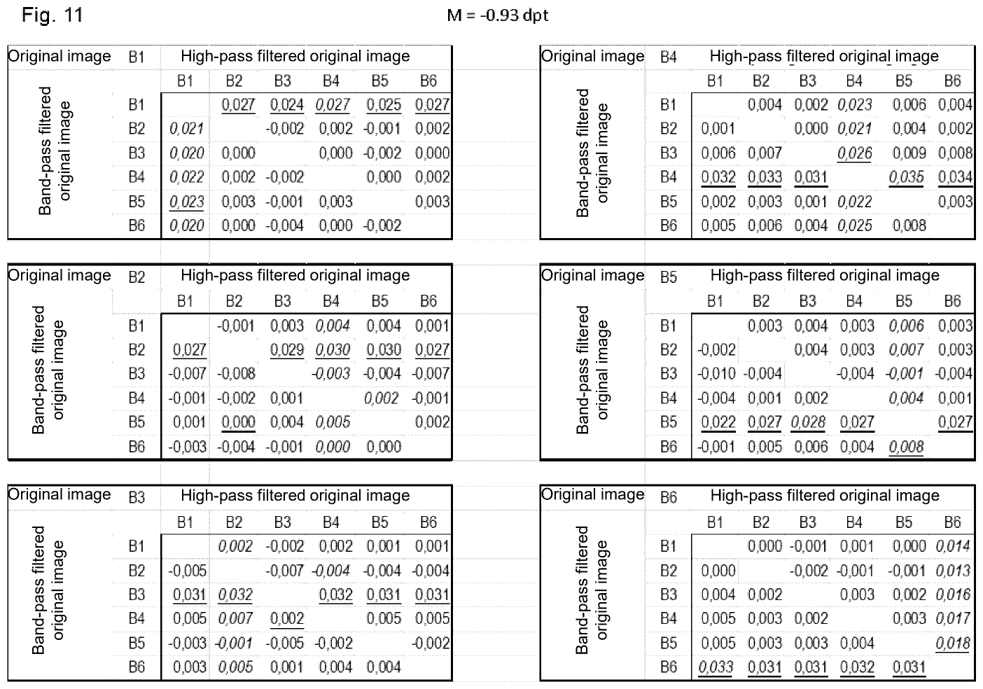

FIG. 11 shows, similar to FIG. 7, tables with calculated similarities of the original images B₁ to B₆ to the combined images shown in FIG. 6. Unlike the tables in FIG. 7, however, the similarity values contained in the tables in FIG. 11 refer to an observation with the optical blur P₂ (M=−0.93 dpt, J₀=−0.03 dpt, J₄₅=−0.02 dpt). The highest values in a row or column are also shown in italics or underlined in the tables in FIG. 11.

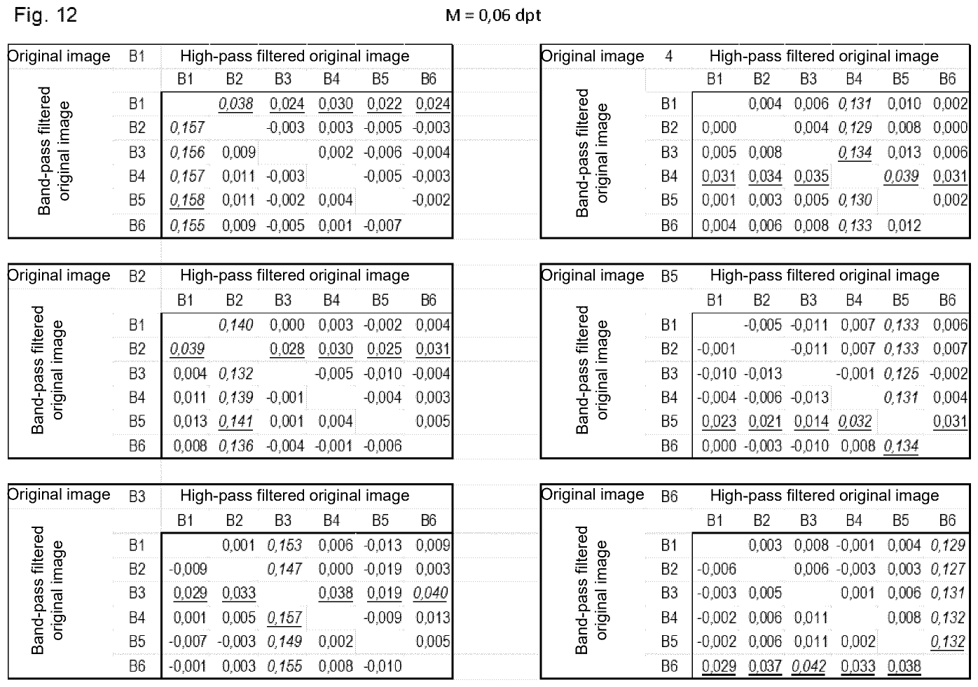

FIG. 12 shows, similar to FIG. 7, tables with calculated similarities of the original images B₁ to B₆ to the combined images shown in FIG. 6. Unlike in the tables in FIG. 7, however, the similarity values contained in the tables in FIG. 12 refer to an observation with the optical blur P₃ (M=0.06 dpt, J₀=−0.03 dpt, J₄₅=−0.02 dpt). The highest values of a row or column are also shown in italics or underlined in the tables in FIG. 12.

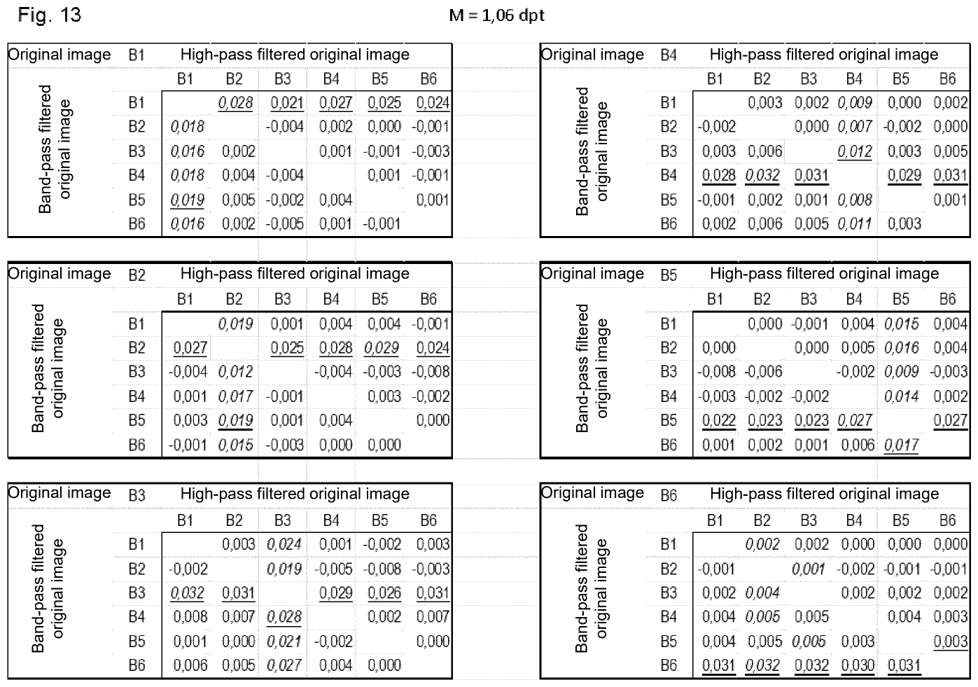

FIG. 13 shows, similar to FIG. 7, tables with calculated similarities of the original images B₁ to B₆ to the combined images shown in FIG. 6. Unlike the tables in FIG. 7, however, the similarity values contained in the tables in FIG. 13 refer to an observation with the optical blur P₄ (M=1.06 dpt, J₀=−0.03 dpt, J₄₅=−0.02 dpt). The highest values in a row or column are also shown in italics or underlined in the tables in FIG. 13.

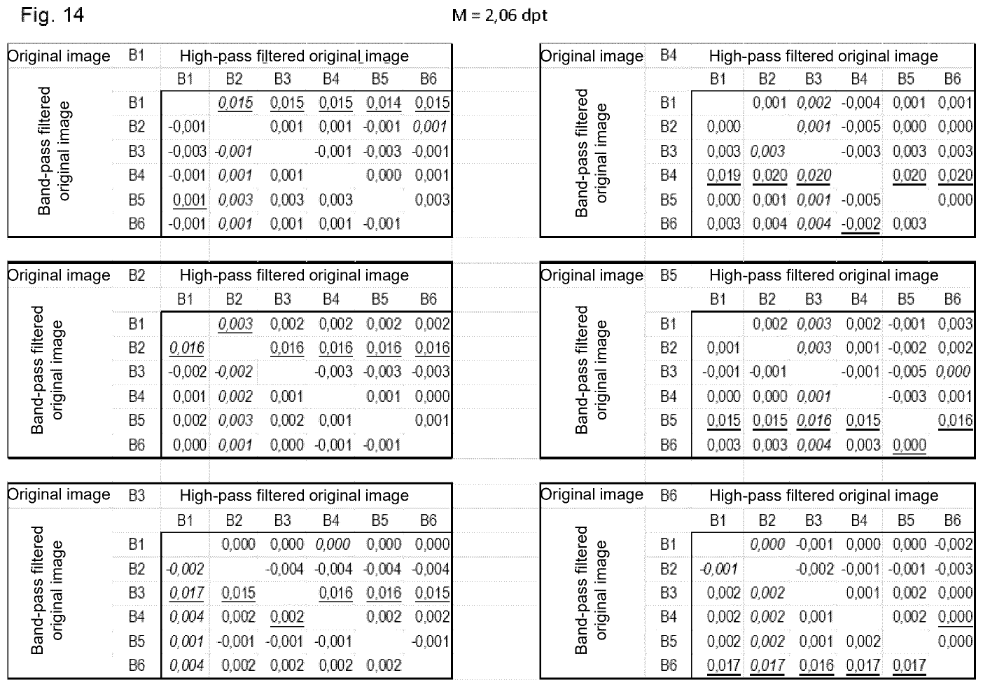

FIG. 14 shows, similar to FIG. 7, tables with calculated similarities of the original images B₁ to B₆ to the combined images shown in FIG. 6. Unlike the tables in FIG. 7, however, the similarity values contained in the tables in FIG. 14 refer to an observation with the optical blur P₅ (M=2.06 dpt, J₀=−0.03 dpt, J₄₅=−0.02 dpt). The highest values in a row or column are also shown in italics or underlined in the tables in FIG. 14.

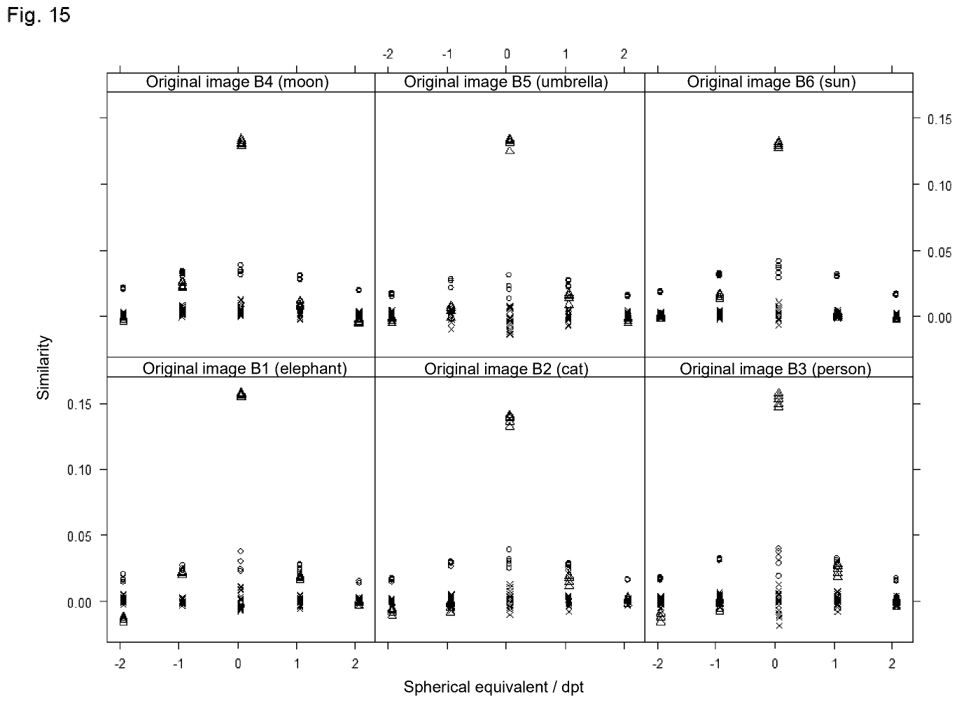

FIG. 15 shows a graphical representation of the similarities from the previous tables in FIGS. 7 and 10 to 14. Each sub-image in FIG. 15 shows the similarity of the original image indicated in the title of the sub-image (“moon”, “umbrella”, “sun”, “elephant”, “cat”, “human”) to the combined filtered images, which are viewed through different optically induced blurs (only the dependence on the spherical equivalent is shown). Triangles or circles represent combined filtered images, for the generation of which the respective original image was high-pass or band-pass filtered. Crosses represent combined filtered images, for the generation of which the original image was not used. The similarity of the original image decreases quickly with a spherical equivalent deviating from 0, or less quickly if the respective original image was high-pass or band-pass filtered to generate the filtered combined image.

### Embodiment for the Definition or Determination of a Similarity:

As already mentioned above, the similarity can be specified in particular by a similarity function S(B₁, B₂) of two images B₁ and B₂. Such a similarity function S(B₁, B₂) can be determined e.g. from the ratio of the mean drop in the cross-correlation function of the images (after background subtraction), DCF(B₁, B₂), and the mean value of the mean drop in the autocorrelation function of the respective images (also after background subtraction), DCF(B₁, B₁) and DCF(B₂, B₂). The similarity function S(B₁, B₂) can therefore be defined as follows:

$$S ( {B}_{1} , {B}_{2} ) = \frac{2 DCF ( {B}_{1} , {B}_{2} )}{DCF ( {B}_{1} , {B}_{1} ) + DCF ( {B}_{2} , {B}_{2} )}$$

It is noted that in the formula specified above, B₁ and B₂ refer to images in general. B₁ and B₂ do not have to be original images, but can be any images, inter alia also filtered images. The mean decrease of the cross-correlation function, DCF(B₁, B₂), or the autocorrelation functions DCF(B₁, B₁) and DCF(B₂, B₂), for a shift of one pixel up, down, right and left can be calculated as follows:

$$DCF ( {B}_{i} , {B}_{j} ) = \frac{1}{4} \{ CF ( {B}_{i} , {B}_{j} ) [ {x}_{0} + 1 , {y}_{0} ] + CF ( {B}_{i} , {B}_{j} ) [ {x}_{0} - 1 , {y}_{0} ] + CF ( {B}_{i} , {B}_{j} ) [ {x}_{0} , {y}_{0} + 1 ] + CF ( {B}_{i} , {B}_{j} ) [ {x}_{0} , {y}_{0} - 1 ] - 4 CF ( {B}_{i} , {B}_{j} ) [ {x}_{0} , {y}_{0} ] \} ,$$

where x₀ and y₀ describe the two-dimensional pixel of the maximum of the correlation function CF (Bᵢ, Bⱼ), which is defined by

$$CF ( {B}_{i} , {B}_{j} ) [ k , l ] = \frac{1}{( M - \vert k \vert ) ( N - \vert l \vert )} {\sum }_{m , n} ( {B}_{i} [ m , n ] - {\overline{B}}_{i} ) ( {B}_{j} [ m + k , n + l ] - {\overline{B}}_{j} )$$

The summation is carried out across all pixels of the image with the positions (m, n). Here, Bᵢ[m, n] denotes the intensity of the i-th image at the position (m, n) and B̅ᵢ denotes the mean value of the intensity across all pixels of the i-th image. Instead of the maximum x₀ and y₀ of the correlation function CF (Pᵢ, Pⱼ), the pixel without displacement can also be selected, i.e. x₀=0 and y₀=0. M and N refer here to the size of the images in pixels (M×N pixels).

Embodiment for the Definition or Quantification of a Similarity Between Two Images Based on the Classification Probability of a Classifier (e.g. Neural Network or Other Machine-Learning Model):

A classifier for quantifying the similarity between two images could be e.g. a deep convolutional neural network, which was pre-trained using existing image databases (e.g. ImageNet), and the last layers of which were trained using a data set containing the original images with their correct assignments (so-called labels) in order to correctly assign the original images. In the embodiment of FIGS. 1a to 3, this would be e.g. the original image of Kermit with the label “Kermit” and the original image of Ms. Piggy with the label “Ms. Piggy”, as well as typically further original images. The artificial neural network is trained with the aim of predicting the label from the original images, i.e. solving a typical classification task. In the embodiment shown in FIGS. 1a to 3, the various possible classes “Kermit” and “Ms. Piggy” belong to would be present in a so-called “one-hot” representation, i.e., for example, “Kermit”=(1, 0, 0, . . . ) and “Ms. Piggy”=(0, 1, 0, 0, . . . ). The vector output by the artificial neural network can therefore be interpreted as a vector of probabilities for classifying the image fed into the input layer into the classes in question (as a “one-hot” representation) using the “loss function” used during training (typically this is the “categorical cross-entropy”).

If e.g. the original image of Kermit is fed into the network, then an output vector (0.96, 0.05, 0.06, 0.01, . . . ) can be output. If the original image of Ms. Piggy is fed into the network, then the output vector (0.03, 0.92, 0.08, 0.02, . . . ) will result. The numerical values used here merely serve as examples. If any other image is fed into the network, then the network will output an output vector the k-th element of which can be understood as the similarity of the k-th original image to the filtered image, or the recognizability of the k-th original image in the filtered image. The arbitrary other image can be a filtered image assigned to a first optically induced blur, in which, in addition to filtering with a filter function, the effect of viewing was simulated by a second optically induced blur. The k-th element of the output vector can be considered as the recognizability of the k-th original image in the filtered image associated with the first optically induced blur when viewed through the second optically induced blur.

1-15. (canceled)

16. A visual object for determining one or more visual properties of a test person, comprising:
at least two different filtered images combined to form a test image,
wherein each of the filtered images represents one of at least two predetermined different original images and is provided with a filter specific to a predetermined optically induced blur.

17. The visual object according to claim 16, wherein each of the filtered images is provided with a filter specific to a predetermined optically induced blur such that on the test image:
when viewed with a first predetermined optically induced blur, a first one of the at least two original images is better recognizable than each of the remaining original images, which differs from the first original image, and
when viewed with a second predetermined optically induced blur, a second one of the at least two original images is better recognizable than each of the remaining original images, which differs from the second original image.

18. The visual object according to claim 16, wherein each of the filtered images is provided with a filter specific to a predetermined optically induced blur such that the filtered image, when viewed with the predetermined specific optically induced blur, has a predetermined similarity and/or a predetermined recognizability to its represented original image.

19. The visual object according to claim 16,
wherein the at least two filtered images comprise a first filtered image F₁(B₁) and a second filtered image F₂(B₂),
wherein the first filtered image F₁(B₁) represents a first original image B₁ filtered with a first filter F₁, and
wherein the second filtered image F₂(B₂) represents a second original image B₂ filtered with a second filter F₂, which differs from the first original image B₁.

20. The visual object according to claim 19,
wherein a similarity S₁₁₁ of the first filtered image F₁(B₁) to its original image B₁ when viewed with a first blur P₁ is greater than a similarity S₂₁₂ of the second filtered image F₂(B₂) to its original image B₂ when viewed with the first blur P₁.

21. The visual object according to claim 20, wherein it applies:
S₁₁₁S₁₂₁, and/or
S₂₂₂>S₁₂₁, and/or
S₂₂₂>S₂₁₂,
where S₁₁₁ is the similarity of the first filtered image F₁(B₁) to its original image B₁ when viewed with the first blur P₁,
where S₂₂₂ is the similarity of the second filtered image F₂(B₂) to its original image B₂ when viewed with a second blur P₂,
where S₁₂₁ is the similarity of the first filtered image F₁(B₁) to its original image B₁ when viewed with the second blur P₂, and
where S₂₁₂ is the similarity of the second filtered image F₂(B₂) to its original image B₂ when viewed with the first blur P₁.

22. The visual object according to claim 21, wherein it further applies:
S₁₁₂<S₁₁₁ and S₁₁₂<S₂₂₂, and/or
S₁₂₂<S₁₁₁ and S₁₂₂<S₂₂₂,
where S₁₁₁ is the similarity of the first filtered image F₁(B₁) to its original image B₁ when viewed with the first blur P₁,
where S₂₂₂ is the similarity of the second filtered image F₂(B₂) to its original image B₂ when viewed with a second blur P₂,
where S₁₁₂ is the similarity of the first filtered image F₁(B₁) to the original image B₂ when viewed with the first blur P₁, and
where S₁₂₂ is the similarity of the first filtered image F₁(B₁) to the original image B₂ when viewed with the second blur P₂.

23. The visual object according to claim 16, wherein the at least two filtered images are arranged next to one another and/or in an overlapping fashion.

24. The visual object according to claim 16,
wherein the filters are adapted to average higher-order aberrations of an eye, or
wherein the filters are adapted to individual higher-order aberrations of at least one eye of the test person.

25. A method for determining one or more visual properties of a test person, comprising:
providing a visual object according to claim 16.

26. The method according to claim 25,
wherein providing the visual object comprises presenting the visual object, and/or
wherein the visual object is presented to the test person.

27. The method according to claim 26, wherein providing a visual object comprises:
providing at least two original images (B₁, B₂); and
generating at least two filtered recognition images by applying filters specific to a predetermined optically induced blur to the at least two original images such that each filtered recognition image is assigned to one of the at least two original images and that when the filtered recognition images are viewed with the predetermined optically induced blur, each filtered recognition image has a predetermined similarity to its assigned original image.

28. The method according to claim 26, wherein providing the visual object comprises determining the filter specific to a predetermined optically induced blur for each original image.

29. The method according to claim 28, wherein the determining the filter specific to a predetermined optically induced blur for each original image comprises calculating an optical transfer function associated with the respective optically induced blur.

30. The method according to claim 26, wherein providing the visual object comprises:
filtering a plurality N of different original images Bᵢ with 1≤i≤N, for a plurality M of optically induced blurs Pⱼ with 1≤j & M, where M and N are each a positive integer with M<N, and
selecting those filtered images Fⱼ(Bᵢ) the similarity Sᵢᵢ of which to the original images Bᵢ exceeds a first threshold value when viewed with an optically induced blur Pᵢ, and the similarity Sᵢⱼ of which to the other original images Bⱼ with j≠i when viewed with an optically induced blur Pᵢ falls below a second threshold value.

31. The method according to claim 30, wherein the selection of filtered images represents an optimization across all subsets with M original images, wherein the optimization is preferably carried out by maximizing a target function.

32. The method according to claim 31, herein the target function is defined as follows:
Σᵢⱼₖwᵢⱼₖ(2δᵢⱼδᵢₖ−1)Sᵢⱼₖ, where
δ represents the Kronecker symbol and wᵢⱼₖ are weights with wᵢⱼₖ≥0 and Σᵢⱼₖ wᵢⱼₖ>0.

33. The method according to claim 26, further comprising:
evaluating a test person's reaction to the visual object.

34. The method according to claim 33, wherein the evaluating comprises:
determining a test person's ametropia, and/or
determining the test person's sensitivity to an optically induced blur, and/or
determining a filter optimal for the test person.

35. A non-transitory computer program product, comprising computer-readable instructions which, when loaded into a memory of a computer and executed by the computer, cause the computer to carry out a method according to claim 26.
